(19) United States
(12) Patent Application Publication
(10) Pub. No.: US 2024/0329073 A1
(43) Pub. Date: Oct. 3, 2024

(54) INTELLIGENT STORAGE SYSTEM FOR SAMPLE REPOSITORY

(71) Applicant: SHANGHAI ORIGINCELL BIOLOGICAL CRYO EQUIPMENT CO., LTD., Shanghai (CN)

(72) Inventors: Jianguo QU, Shanghai (CN); Weiguang CAO, Shanghai (CN)

(73) Assignee: SHANGHAI ORIGINCELL BIOLOGICAL CRYO EQUIPMENT CO., LTD., Shanghai (CN)

(21) Appl. No.: 18/573,420

(22) PCT Filed: Mar. 25, 2022
(86) PCT No.: PCT/CN2022/082897

§ 371 Date: Dec. 21, 2023

(30) Foreign Application Priority Data
Jul. 8, 2021 (CN) 202110774267.6

(51) Int. Cl. G01N 35/10; G01N 35/04

(52) U.S. Cl. CPC G01N 35/10; G01N 35/04; G01N 2035/0406; G01N 2035/0465; G01N 2035/1053

(57) ABSTRACT

An intelligent storage system for a sample repository is provided, including at least one group of storage modules (1) and at least one group of extraction modules (2). The storage module (1) includes at least two storage tanks (3). Access ports (4) are formed in upper end surfaces of the storage tanks (3), and positions of the access ports (4) of two corresponding storage tanks (3) correspond to each other. The extraction modules (2) are disposed above the storage modules (1), and are capable of extracting or storing cryopreservation tubes and/or storage racks in the storage tanks (3) via the access ports (4). The storage modules (1) can be connected by means of multiple types of transfer devices to quickly transfer and transport samples and to provide an emergency solution.

### TECHNICAL FIELD

The present invention relates to the field of biological sample storage, and in particular, to an intelligent storage system for a sample repository.

### BACKGROUND

In the storage of biological samples, especially storage and extraction of cryopreservation tubes, one storage device usually corresponds to one extraction module, and samples of a plurality of devices cannot be quickly and efficiently transferred. In this case, the cost of use is increased, and the weight of a product is also significantly increased, which reduces use experience of users.

In addition, in an extraction process, a tube picking device or a storage rack grab extracts a storage rack or a sample tube from a liquid nitrogen tank and then performs transfer. In this case, the sample tube is directly exposed to room temperature, easily causing damage to a sample.

### SUMMARY OF THE INVENTION

An objective of the present invention is to provide an intelligent storage system for a sample repository, which resolves the problem that grabbing cannot be performed between a plurality of storage devices by using one type of extraction module and samples cannot be quickly and efficiently transferred between the plurality of storage devices.

The following technical solution is adopted to resolve the technical problem in the present invention:

An intelligent storage system for a sample repository includes at least one group of storage modules and at least one group of extraction modules, where
the storage module includes at least two storage tanks, access ports are formed in upper end surfaces of the storage tanks, and positions of the access ports of the two corresponding storage tanks correspond to each other; and
the extraction modules are disposed above the storage modules, and are capable of extracting or storing cryopreservation tubes and/or storage racks in the storage tanks via the access ports.

Preferably, the access port in the storage tank is formed in a side of the upper end surface of the storage tank, and the access ports of the two storage tanks that correspond to each other are opposite and close to each other.

Preferably, the extraction module includes an X-axis extraction arm set, a Y-axis extraction arm set, and a grabbing apparatus, and the grabbing apparatus is capable of sucking or grabbing a cryopreservation tube and/or grabbing a storage rack in the storage tank; and
the X-axis extraction arm set is capable of driving the grabbing apparatus to move along an X axis, and the Y-axis extraction arm set is capable of driving the grabbing apparatus to move along a Y axis.

Preferably, the system includes a group A of storage modules, a group A of extraction modules, a group B of storage modules, and a group B of extraction modules, where
the group A of storage modules and the group B of storage modules are disposed side by side, the group A of extraction modules are disposed above and act on the group A of storage modules, and the group B of extraction modules are disposed above and act on the group B of storage modules; and
a penetrating transfer channel is directly provided between the group A of extraction modules and the group B of extraction modules, and the transfer channel is capable of transferring a transport tank.

Preferably, the transfer channel includes a transmission member that performs transmission along a side-by-side direction of the group A of storage modules and the group B of storage modules.

Preferably, a plurality of lifting channels are formed in the transmission member; and the extraction module further includes a lifting mechanism, the lifting mechanism is disposed between the two storage tanks of the storage module, and the lifting mechanism is capable of lifting the transport tank through the lifting channel.

Preferably, the lifting mechanism includes an elevator platform and a driving electric cylinder, the elevator platform is disposed between the two storage tanks of the storage module, one end of the elevator platform is disposed below the lifting channel, the other end of the elevator platform extends outward and is vertically disposed, and the driving electric cylinder is vertically disposed and is connected to an extension end of the elevator platform; and
the driving electric cylinder is capable of driving the elevator platform to ascend and descend in a vertical direction.

Preferably, the Y-axis extraction arm set includes a Y-axis bearing track and a Y-axis movement mechanism, and the Y-axis bearing track includes a bearing track main shaft and a bearing track sub-shaft;
the bearing track main shaft and the bearing track sub-shaft are disposed in parallel and symmetrically at upper ends on two sides of the storage module, the Y-axis movement mechanism is disposed on the bearing track main shaft and the bearing track sub-shaft and is capable of sliding along the bearing track main shaft and the bearing track sub-shaft, and the Y-axis movement mechanism is connected to the X-axis extraction arm set and is capable of driving the X-axis extraction arm set to move along the Y axis direction; and
the Y axis is perpendicular to a direction along which the storage tanks are set side by side.

Preferably, the X-axis extraction arm set includes an X-axis bearing track and an X-axis movement mechanism; two ends of the X-axis bearing track are vertically disposed on the Y-axis bearing track and are connected to the Y-axis movement mechanism, the X-axis movement mechanism is disposed on the X-axis bearing track and is capable of sliding along the X-axis bearing track, and the X-axis movement mechanism is connected to the grabbing apparatus and is capable of driving the grabbing apparatus to move in an X axis direction; and
the X axis is parallel to a direction along which the storage tanks are set side by side.

Preferably, the X-axis extraction arm set includes an X-axis sliding track and a sliding mechanism; the X-axis sliding track is vertically disposed below the Y-axis bearing track with two ends extending to connect the group A of storage modules and the group B of storage modules in series, the X-axis sliding track is connected to the Y-axis movement mechanism, the sliding mechanism is disposed on the X-axis sliding track and is capable of sliding along the X-axis sliding track, and the sliding mechanism is connected to the grabbing apparatus and is capable of driving the grabbing apparatus to move in an X axis direction; and
the X axis is parallel to a direction along which the storage tanks are set side by side.

Preferably, the sliding mechanism includes a wheel set, a connecting rotating shaft, and a sliding track bearing support; and the wheel set is symmetrically disposed in the X-axis sliding track and is capable of sliding along the X-axis sliding track, the sliding track bearing support is disposed on the X-axis sliding track and is connected to the wheel set, and the connecting rotating shaft is disposed on the sliding track bearing support and is connected to the grabbing apparatus.

Preferably, the grabbing apparatus includes a cryopreservation tube extraction mechanism, a storage rack grabbing mechanism, and a connecting mechanism; an upper end of the connecting mechanism is connected to the X-axis extraction arm set, and a lower end is connected to the storage rack grabbing mechanism and the cryopreservation tube extraction mechanism; and
the X-axis extraction arm set is capable of grabbing a cryopreservation tube or a storage rack in the storage tank and is capable of driving the cryopreservation tube or the storage rack to move along the X axis, and the Y-axis extraction arm set is capable of allowing for grabbing a cryopreservation tube or a storage rack in the storage tank and is capable of driving the cryopreservation tube or the storage rack to move along the Y axis.

Preferably, the system further includes a module protection housing, where the module protection housing is disposed on the storage modules and is disposed outside the extraction modules; and
a main control interface is further provided on the module protection housing.

Preferably, the storage module further includes a valve assembly, and the valve assembly is disposed on a side surface of the module protection housing.

Preferably, the valve assembly includes a coil liquid-adding mechanism, and the coil liquid-adding mechanism is disposed on a side surface of the module protection housing.

The present invention has the following beneficial effects:

1. Since two access ports are provided in a storage module in a face-to-face manner, and only one extraction module is required above the storage module, it is possible that two storage tanks use only one extraction module, thereby significantly improving the working efficiency and reducing the cost of use.

2. By means of a combination of two storage tanks, various types of biological samples or cells can be separately stored in one same storage module, so that cross contamination is prevented more effectively, and switching and protection can be performed in time when an accident occurs, thereby ensuring the safety of biological samples or cells.

3. The storage modules can be connected by means of multiple types of transfer devices to quickly transfer and transport samples and to provide an emergency solution. When there is a small amount of liquid nitrogen in one storage tank, samples can be transferred to a nearby storage tank, thereby minimizing a risk, and improving the working efficiency.

4. The cryopreservation tube extraction mechanism and the storage rack grabbing mechanism directly grab a cryopreservation tube and a storage rack via the access port, to provide a heat preservation solution, thereby reducing a damage probability of samples during extraction and transport.

5. The storage module can meet long term and short term storage requirements, and frequent use and long-term storage are appropriately separated, which helps to mitigate mutual impact in a storage process.

### BRIEF DESCRIPTION OF THE DRAWINGS

FIG. 1 is an overall schematic structural diagram according to the present invention;

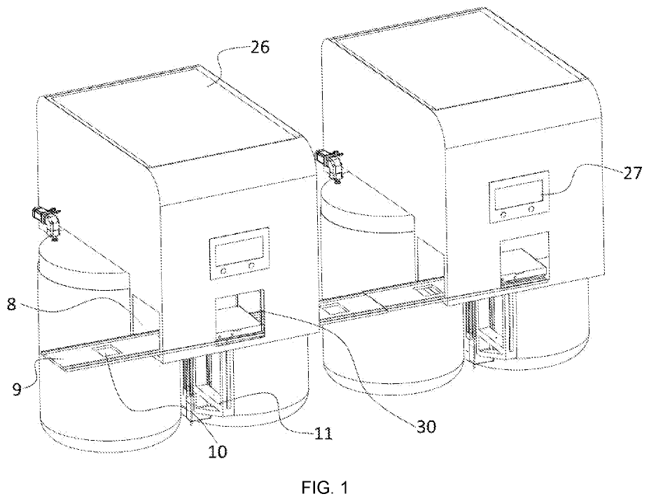

FIG. 2 is a schematic structural diagram in a module protection housing according to the present invention;

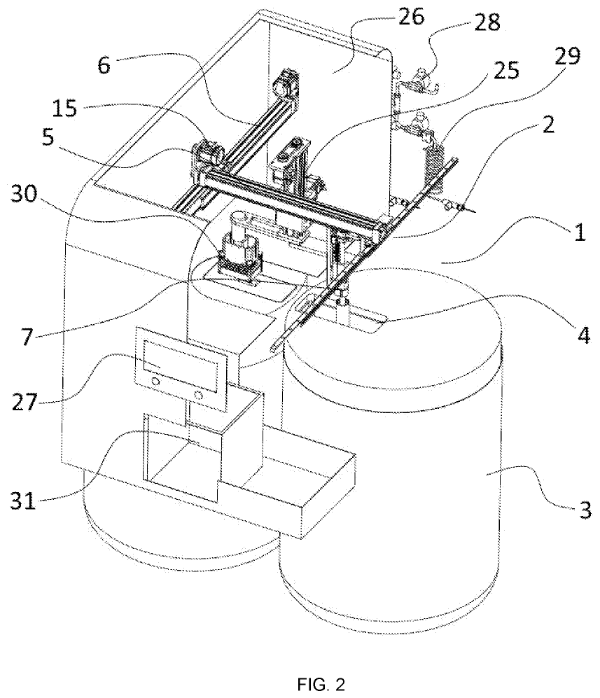

FIG. 3 is a schematic structural diagram of an extraction module according to the present invention;

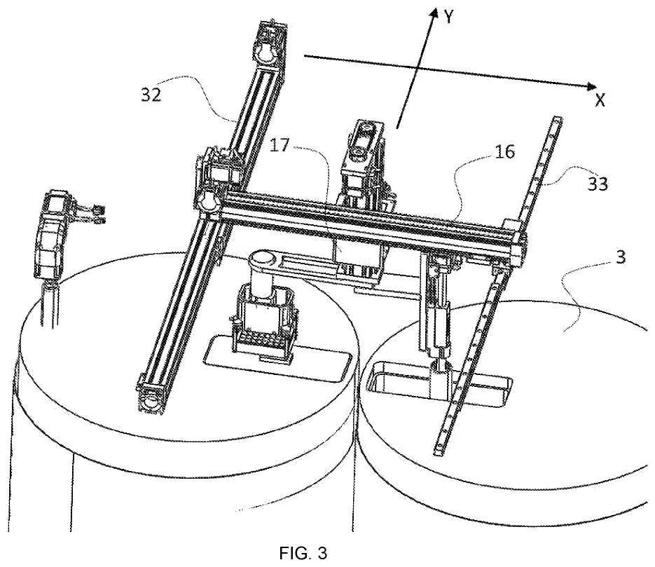

FIG. 4 is a schematic structural diagram according to Embodiment 2 of the present invention;

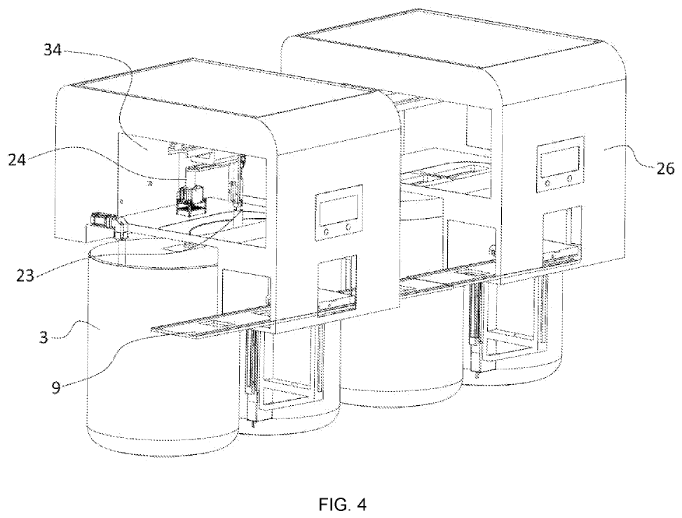

FIG. 5 is a schematic structural diagram in a module protection housing according to Embodiment 2 of the present invention; and

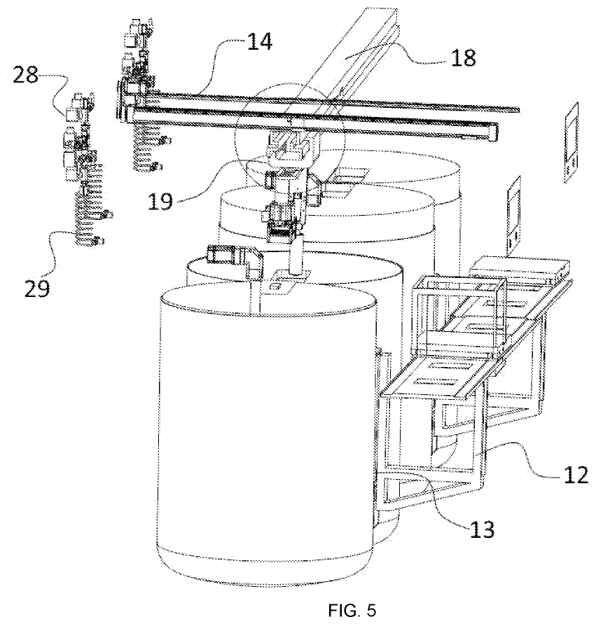

FIG. 6 is a schematic structural diagram of a sliding mechanism according to Embodiment 2 of the present invention.

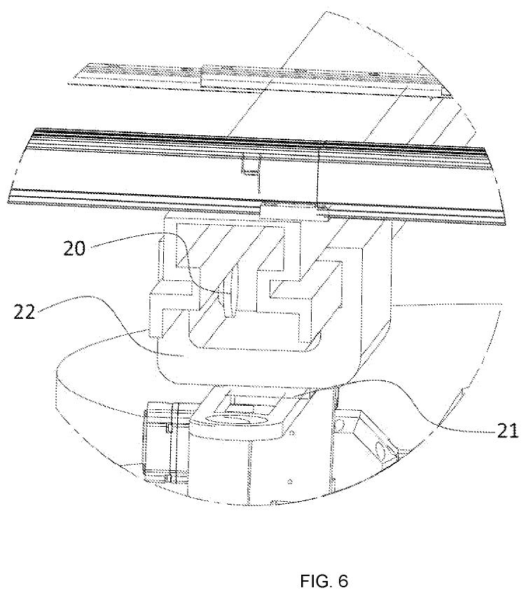

### REFERENCE NUMERALS

storage module; 2-extraction module; 3-storage tank; 4-access port; 5-X-axis extraction arm set; 6-Y-axis extraction arm set; 7-grabbing apparatus; 8-transfer channel; 9-transmission member; 10-lifting channel; H-lifting mechanism; 12-elevator platform; 13-driving electric cylinder; 14-Y-axis bearing track; 15-Y-axis movement mechanism; 16-X-axis bearing track; 17-X-axis movement mechanism; 18-X-axis sliding track; 19-sliding mechanism; 20-wheel set; 21-connecting rotating shaft; 22-sliding track bearing support; 23-cryopreservation tube extraction mechanism; 24-storage rack grabbing mechanism; 25-connecting mechanism; 26-module protection housing; 27-main control interface; 28-valve assembly; 29-coil liquid-adding mechanism; 30-transport window; 31-transport tank; 32-bearing track main shaft; 33-bearing track sub-shaft; and 34-upper transfer channel.

### DETAILED DESCRIPTION

The technical solutions of the present invention are further described below with reference to the embodiments and the accompanying drawings.

### Embodiment 1

This embodiment provides an intelligent storage system for a sample repository, including at least one group of storage modules 1 and at least one group of extraction modules 2.

The storage module 1 includes at least two storage tanks 3, the storage tank 3 is provided with an access port 4 in an upper end surface, and positions of the access ports 4 of the two corresponding storage tanks 3 correspond to each other.

The extraction modules 2 are disposed above the storage modules 1, and are capable of extracting or storing cryopreservation tubes and/or storage racks in the storage tanks 3 via the access ports 4.

During storage and extraction of a cryopreservation tube for biological samples, generally one storage tank 3 corresponds to one extraction module 2. In this case, the cost for transport between different storage tanks 3 is high, and the cryopreservation tube can be easily damaged as it is exposed to room temperature. The present invention discloses a storage module 1 consisting of two storage tanks 3. The storage tanks 3 are disposed side by side, and the access ports 4 are formed in upper end surfaces of the storage tanks 3. The two access ports 4 are close to each other, to facilitate storage and grabbing operations of the extraction module 2 between different storage tanks 3, so that the working efficiency of the extraction module 2 can be improved, and the cost can be reduced.

In a further implementation of the present invention, the access port 4 in the storage tank 3 is formed in one side of the upper end surface of the storage tank 3, and the access ports 4 of the two storage tanks 3 that correspond to each other are opposite and close to each other. The closer the access ports 4 are to each other, the more convenient it is for the extraction module 2 to operate between different storage tanks 3, and the access ports 4 can be set face-to-face in different directions. In the present invention, the access ports 4 are set face-to-face in the radial direction and parallel to a side-by-side arrangement direction of the storage tanks 3, to maximize the convenience of the extraction module 2 in storing or extracting cryopreservation tubes on the storage module 1.

In a further implementation of the present invention, the extraction module 2 includes an X-axis extraction arm set 5, a Y-axis extraction arm set 6, and a grabbing apparatus 7. The grabbing apparatus 7 is capable of sucking or grabbing a cryopreservation tube and/or grabbing a storage rack in the storage tank 3.

The Y-axis extraction arm set 6 is connected to the X-axis extraction arm set 5, and is capable of driving the X-axis extraction arm set 5 to move along a Y axis. The X-axis extraction arm set 5 is connected to the grabbing apparatus 7, and is capable of driving the grabbing apparatus 7 to move along an X axis. The grabbing apparatus 7 can move along the X axis and the Y axis above the storage tank 3, to grab storage racks and/or cryopreservation tubes via the access ports 4 at different positions, thereby improving the working efficiency.

When the access ports 4 in the two storage tanks 3 are closer to each other, the X-axis extraction arm set 5 and the Y-axis extraction arm set 6 can grab a storage rack and/or a cryopreservation tube with fewer movements, thereby saving time and improving efficiency. Therefore, the face-to-face radial arrangement of the access ports 4 in a side-by-side direction of the storage tanks 3 has the highest efficiency.

In a further implementation of the present invention, the system includes a group A of storage modules, a group A of extraction modules, a group B of storage modules, and a group B of extraction modules.

The group A of storage modules and the group B of storage modules are disposed side by side, the group A of extraction modules are disposed above and act on the group A of storage modules, and the group B of extraction modules are disposed above and act on the group B of storage modules.

A penetrating transfer channel 8 is directly provided between the group A of extraction modules and the group B of extraction modules, and the transfer channel 8 is capable of transferring a transport tank 31. The transport tank 31 may transit and transport a cryopreservation tube. Liquid nitrogen is stored in the transport tank 31 for heat preservation in a transport process of the cryopreservation tube.

The number of storage modules in the group B of storage modules is at least one, and the group B of storage modules are sequentially disposed side by side. The transfer channel 8 connects the group A of storage modules and the group B of storage modules in series, to quickly transfer and transport samples between different storage tanks 3.

In a further implementation of the present invention, the transfer channel 8 includes a transmission member 9 that performs transmission along a side-by-side direction of the group A of storage modules and the group B of storage modules.

The transmission member 9 transports the transport tank 31 in a side-by-side horizontal direction of the storage modules 1. The transmission member 9 may convey the transport tank 31 through a conveyor belt, a convey plate or a conveyor track. There are many implementations of the transmission member 9, and details are not described again.

In a further implementation of the present invention, a plurality of lifting channels 10 are formed in the transmission member 9; a transport window 30 is formed in a side surface of the transfer channel 8; and the extraction module 2 further includes a lifting mechanism 11, the lifting mechanism 11 is disposed between the two storage tanks 3 of the storage module 1, and the lifting mechanism 11 is capable of lifting the transport tank 31 through the lifting channel 10.

The transport tank 31 is delivered to the transfer channel 8 from the transport window 30 formed in the side surface of the transfer channel 8, and is conveyed to an upper end of the lifting channel 10 via the transfer channel 8. The transport tank 31 is then lifted by the lifting mechanism 11 to an upper end of the storage tank 3 through the lifting channel 10, to store or extract a cryopreservation tube. After operations are ended, the lifting mechanism 11 descends to deliver the transport tank 31 back to the transfer channel 8. A cryopreservation tube in the transport tank 31 can be quickly transported and transferred between different storage modules 1 by the transmission member 9 in the transfer channel 8, thereby improving the working efficiency.

In a further implementation of the present invention, the lifting mechanism 11 includes an elevator platform 12 and a driving electric cylinder 13. The elevator platform 12 is disposed between the two storage tanks 3 of the storage module 1, one end of the elevator platform 12 is disposed below the lifting channel 10, the other end of the elevator platform 12 extends outward and is vertically disposed, and the driving electric cylinder 13 is vertically disposed and is connected to an extension end of the elevator platform 12. The driving electric cylinder 13 is disposed on a side of the transfer channel 8 close to the storage tank 3, so that the overall space of this system can be reduced. The driving electric cylinder 13 is capable of driving the elevator platform 12 to ascend or descend in a vertical direction. The lifting mechanism 11 resolves the problem of transport between the storage tank 3 and the transfer channel 8, thereby significantly improving the efficiency of quickly transferring samples.

In a further implementation of the present invention, the Y-axis extraction arm set 6 includes a Y-axis bearing track 14 and a Y-axis movement mechanism 15. The Y-axis bearing track 14 includes a bearing track main shaft 32 and a bearing track sub-shaft 33. The bearing track main shaft 32 and the bearing track sub-shaft 33 are disposed in parallel and symmetrically above two sides of the storage module 1, the Y-axis movement mechanism 15 is disposed on the bearing track main shaft 32 and the bearing track sub-shaft 33 and is capable of sliding along the bearing track main shaft 32 and the bearing track sub-shaft 33, and the Y-axis movement mechanism 15 is connected to the X-axis extraction arm set 5 and is capable of driving the X-axis extraction arm set 5 to move along the Y axis direction. The Y-axis is a horizontal direction perpendicular to the direction in which the storage tanks 3 are set side by side.

The Y-axis extraction arm set 6 is used to transport a cryopreservation tube between the transport tank 31 and the storage tank 3. The grabbing apparatus 7 is driven by the Y-axis extraction arm set 6 to move in the horizontal direction perpendicular to the direction in which the storage tanks 3 are set side by side, to facilitate the grabbing operations of the grabbing apparatus.

The Y-axis movement mechanism 15 is provided with a strap to be fastened to the X-axis extraction arm set 5, and is provided with a sliding member. While sliding on the bearing track main shaft 32 and the bearing track sub-shaft 33, the Y-axis movement mechanism drives the X-axis extraction arm set 5 to move horizontally along the Y axis, and the Y-axis movement mechanism 15 may be implemented as a sliding block, a sliding wheel, or the like.

In a further implementation of the present invention, the X-axis extraction arm set 5 includes an X-axis bearing track 16 and an X-axis movement mechanism 17. Two ends of the X-axis bearing track 16 are vertically disposed on the bearing track main shaft 32 and the bearing track sub-shaft 33 and are connected to the Y-axis movement mechanism 15. The X-axis movement mechanism 17 is disposed on the X-axis bearing track 16 and is capable of sliding along the X-axis bearing track 16, and the X-axis movement mechanism 17 is connected to the grabbing apparatus 7 and is capable of driving the grabbing apparatus 7 to move in an X axis direction. The X axis is parallel to the direction in which the storage tanks 3 are set side by side.

The two ends of the X-axis bearing track 16 are respectively disposed on the bearing track main shaft 32 and the bearing track sub-shaft 33 to move horizontally in one storage module 1, and the grabbing apparatus 7 can be driven to move in an X axis direction, that is, to move between the access ports 4 of the two storage tanks 3, thereby performing grabbing and storage operations.

In a further implementation of the present invention, the grabbing apparatus 7 includes a cryopreservation tube extraction mechanism 23, a storage rack grabbing mechanism 24, and a connecting mechanism 25; an upper end of the connecting mechanism 25 is connected to the X-axis extraction arm set 5, and a lower end of the connecting mechanism 25 is connected to the storage rack grabbing mechanism 24 and the cryopreservation tube extraction mechanism 23.

The X-axis extraction arm set 5 is capable of grabbing a cryopreservation tube or a storage rack in the storage tank 3 and is capable of driving the cryopreservation tube or the storage rack to move along the X axis to grab the cryopreservation tube and/or the storage rack, to transport samples between different storage tanks 3; and the Y-axis extraction arm set 6 is capable of grabbing a cryopreservation tube or a storage rack in the storage tank 3 and is capable of driving the cryopreservation tube or the storage rack to move along the Y axis to transport samples between the storage tank 3 and the transport tank 31, to transport samples between different storage modules 1, thereby significantly improving the working efficiency.

In a further implementation of the present invention, the system further includes a module protection housing 26. The module protection housing 26 is disposed above the storage module 1 and covers the extraction module 2. Two ends of the Y-axis bearing track 14 are both fastened on an inner wall of the module protection housing 26, and the lifting mechanism 11 is fastened at a lower end of the module protection housing 26. The module protection housing 26 fastens the extraction module 2, and protects a cryopreservation tube extracted by the grabbing apparatus 7 from losing heat too quickly, thereby implementing heat preservation.

A main control interface 27 is further provided on the module protection housing 26. The main control interface 27 is disposed on another side of the transfer channel 8 opposite to the storage tank 3. The main control interface 27 may control the transmission member 9 to start or stop transferring the transport tank 31.

In a further implementation of the present invention, the storage module 1 further includes a valve assembly 28. The valve assembly 28 is disposed on a side surface of the module protection housing 26. The valve assembly 28 may inject liquid nitrogen into the storage tank 3, to maintain a temperature in the tank.

In a further implementation of the present invention, the valve assembly 28 includes a coil liquid-adding mechanism 29. The coil liquid-adding mechanism 29 is disposed on the side surface of the module protection housing 26. The coil liquid-adding mechanism 29 is heated to inject sprayed liquid nitrogen into the storage tank 3, to keep liquid nitrogen from being directly poured at one position to cause temperature non-uniformity. If the temperature in the storage tank 3 is excessively low, a heating wire outside the coil liquid-adding mechanism 29 may be turned on to reach an appropriate storage temperature.

### Embodiment 2

This embodiment provides an intelligent storage system for a sample repository. This embodiment differs from Embodiment 2 in that:

The X-axis extraction arm set 5 includes an X-axis sliding track 18 and a sliding mechanism 19. The X-axis sliding track 18 is vertically disposed at lower ends of the bearing track main shaft 32 and the bearing track sub-shaft 33, with two ends extending to connect the group A of storage modules and the group B of storage modules in series. The X-axis sliding track 18 is connected to the Y-axis movement mechanism 15, the sliding mechanism 19 is disposed on the X-axis sliding track 18 and is capable of sliding along the X-axis sliding track 18, and the sliding mechanism 19 is connected to the grabbing apparatus 7 and is capable of driving the grabbing apparatus 7 to move in an X axis direction. The X axis is parallel to the direction in which the storage tanks 3 are set side by side.

Compared with that the X-axis bearing track 16 moves horizontally along the Y-axis bearing track 14 in Embodiment 1, two ends of the X-axis sliding track 18 extend and may connect in series all the group A of storage modules and the group B of storage modules that are disposed side by side, to transfer samples by multiple types of transfer devices between different storage modules 1, thereby significantly improving the transfer efficiency.

In a further implementation of the present invention, the sliding mechanism 19 includes a wheel set 20, a connecting rotating shaft 21, and a sliding track bearing support 22. The wheel set 20 is symmetrically disposed in the X-axis sliding track 18 and is capable of sliding along the X-axis sliding track 18, the sliding track bearing support 22 is disposed on the X-axis sliding track 18 and is connected to the wheel set 20, and the connecting rotating shaft 21 is disposed on the sliding track bearing support 22 and is connected to the grabbing apparatus 7.

The sliding track bearing support 22 slides on the X-axis sliding track 18 through the wheel set 20, a lower end of the connecting rotating shaft 21 is connected to the grabbing apparatus 7 to drive the grabbing apparatus 7 to move along the X-axis sliding track 18, to implement grabbing and storage between different storage modules 1 by the grabbing apparatus 7.

In a further implementation of the present invention, an upper transfer channel 34 is provided in the module protection housing 26. The upper transfer channel 34 is provided above the storage tank 3 and accommodates the extraction module 2. The upper transfer channel 34 and the transfer channel 8 are provided in the same direction. The X-axis sliding track 18 passes through the upper transfer channel 34 to drive the grabbing apparatus 7 to transport samples between different storage modules 1, thereby significantly improving the working efficiency.

The foregoing sequence of the embodiments are merely for the convenience of description, and do not imply the preference among the embodiments.

Finally, it should be noted that the foregoing embodiments are merely intended for describing the technical solutions of the present invention rather than limiting the present invention. Although the present invention is described in detail with reference to the foregoing embodiments, persons of ordinary skill in the art should understand that they may still make modifications to the technical solutions described in the foregoing embodiments or make equivalent replacements to some the technical features thereof, without departing from the scope of the technical solutions of the embodiments of the present invention.

1. An intelligent storage system for a sample repository, comprising at least one group of storage modules and at least one group of extraction modules, wherein
each of the storage modules comprises at least two storage tanks, each of the storage tanks is provided with an access port in an upper end surface, and positions of access ports of two corresponding storage tanks correspond to each other; and
the extraction modules are disposed above the storage modules, and are capable of extracting or storing cryopreservation tubes and/or storage racks in the storage tanks via the access ports.

2. The intelligent storage system for a sample repository according to claim 1, wherein the access port in each of the storage tanks is formed in a side of the upper end surface of said storage tank, and access ports of two storage tanks that correspond to each other are opposite and close to each other.

3. The intelligent storage system for a sample repository according to claim 2, wherein each of the extraction modules comprises an X-axis extraction arm set, a Y-axis extraction arm set, and a grabbing apparatus, wherein the grabbing apparatus is capable of sucking or grabbing a cryopreservation tube and/or grabbing a storage rack in the storage tanks; and
the X-axis extraction arm set is capable of driving the grabbing apparatus to move along an X axis, and the Y-axis extraction arm set is capable of driving the grabbing apparatus to move along a Y axis.

4. The intelligent storage system for a sample repository according to claim 1, comprising a group A of storage modules, a group A of extraction modules, a group B of storage modules, and a group B of extraction modules, wherein
the group A of storage modules and the group B of storage modules are disposed side by side, the group A of extraction modules are disposed above and act on the group A of storage modules, and the group B of extraction modules are disposed above and act on the group B of storage modules; and
a penetrating transfer channel is directly provided between the group A of extraction modules and the group B of extraction modules, and the transfer channel is capable of transferring a transport tank.

5. The intelligent storage system for a sample repository according to claim 4, wherein the transfer channel comprises a transmission member for performing transmission along a side-by-side direction of the group A of storage modules and the group B of storage modules.

6. The intelligent storage system for a sample repository according to claim 5, wherein a plurality of lifting channels are formed in the transmission member; and
each of the extraction modules further comprises a lifting mechanism, the lifting mechanism is disposed between two storage tanks of each of the storage modules, and the lifting mechanism is capable of lifting the transport tank through the plurality of lifting channel.

7. The intelligent storage system for a sample repository according to claim 6, wherein the lifting mechanism comprises an elevator platform and a driving electric cylinder, the elevator platform is disposed between the two storage tanks of each of the storage modules, one end of the elevator platform is disposed below the lifting channel, the other end of the elevator platform extends outward and is vertically disposed, and the driving electric cylinder is vertically disposed and is connected to an extension end of the elevator platform; and
the driving electric cylinder is capable of driving the elevator platform to ascend or descend in a vertical direction.

8. The intelligent storage system for a sample repository according to claim 3, wherein the Y-axis extraction arm set comprises a Y-axis bearing track and a Y-axis movement mechanism, and the Y-axis bearing track comprises a bearing track main shaft and a bearing track sub-shaft;
the bearing track main shaft and the bearing track sub-shaft are disposed in parallel and symmetrically above two sides of one of the storage modules, the Y-axis movement mechanism is disposed on the bearing track main shaft and the bearing track sub-shaft and is capable of sliding along the bearing track main shaft and the bearing track sub-shaft, and the Y-axis movement mechanism is connected to the X-axis extraction arm set and is capable of driving the X-axis extraction arm set to move along the Y axis direction; and
the Y axis is perpendicular to a direction along which the storage tanks are set side by side.

9. The intelligent storage system for a sample repository according to claim 8, wherein the X-axis extraction arm set comprises an X-axis bearing track and an X-axis movement mechanism;
two ends of the X-axis bearing track are vertically disposed on the Y-axis bearing track and are connected to the Y-axis movement mechanism, the X-axis movement mechanism is disposed on the X-axis bearing track and is capable of sliding along the X-axis bearing track, and the X-axis movement mechanism is connected to the grabbing apparatus and is capable of driving the grabbing apparatus to move in an X axis direction; and
the X axis is parallel to a direction along which the storage tanks are set side by side.

10. The intelligent storage system for a sample repository according to claim 8, wherein the X-axis extraction arm set comprises an X-axis sliding track and a sliding mechanism;
the X-axis sliding track is vertically disposed below the Y-axis bearing track with two ends extending to connect the group A of storage modules and the group B of storage modules in series, the X-axis sliding track is connected to the Y-axis movement mechanism, the sliding mechanism is disposed on the X-axis sliding track and is capable of sliding along the X-axis sliding track, and the sliding mechanism is connected to the grabbing apparatus and is capable of driving the grabbing apparatus to move in an X axis direction; and
the X axis is parallel to a direction along which the storage tanks are set side by side.

11. The intelligent storage system for a sample repository according to claim 10, wherein the sliding mechanism comprises a wheel set, a connecting rotating shaft, and a sliding track bearing support; and
the wheel set is symmetrically disposed in the X-axis sliding track and is capable of sliding along the X-axis sliding track, the sliding track bearing support is disposed on the X-axis sliding track and is connected to the wheel set, and the connecting rotating shaft is disposed on the sliding track bearing support and is connected to the grabbing apparatus.

12. The intelligent storage system for a sample repository according to claim 9, wherein the grabbing apparatus comprises a cryopreservation tube extraction mechanism, a storage rack grabbing mechanism, and a connecting mechanism;
an upper end of the connecting mechanism is connected to the X-axis extraction arm set, and a lower end of the connecting mechanism is connected to the storage rack grabbing mechanism and the cryopreservation tube extraction mechanism; and
the X-axis extraction arm set is capable of grabbing a cryopreservation tube or a storage rack in the storage tank and is capable of driving the cryopreservation tube or the storage rack to move along the X axis, and the Y-axis extraction arm set is capable of allowing for grabbing a cryopreservation tube or a storage rack in the storage tank and is capable of driving the cryopreservation tube or the storage rack to move along the Y axis.

13. The intelligent storage system for a sample repository according to claim 12, further comprising a module protection housing, wherein
the module protection housing is disposed on the storage modules and is disposed outside the extraction modules; and
a main control interface is further provided on the module protection housing.

14. The intelligent storage system for a sample repository according to claim 13, wherein each of the storage modules further comprises a valve assembly, and the valve assembly is disposed on a side surface of the module protection housing.

15. The intelligent storage system for a sample repository according to claim 14, wherein the valve assembly comprises a coil liquid-adding mechanism, and the coil liquid-adding mechanism is disposed on a side surface of the module protection housing.

16. The intelligent storage system for a sample repository according to claim 5, wherein the Y-axis extraction arm set comprises a Y-axis bearing track and a Y-axis movement mechanism, and the Y-axis bearing track comprises a bearing track main shaft and a bearing track sub-shaft;
the bearing track main shaft and the bearing track sub-shaft are disposed in parallel and symmetrically above two sides of one of the storage modules, the Y-axis movement mechanism is disposed on the bearing track main shaft and the bearing track sub-shaft and is capable of sliding along the bearing track main shaft and the bearing track sub-shaft, and the Y-axis movement mechanism is connected to the X-axis extraction arm set and is capable of driving the X-axis extraction arm set to move along the Y axis direction; and
the Y axis is perpendicular to a direction along which the storage tanks are set side by side.

17. The intelligent storage system for a sample repository according to claim 6, wherein the Y-axis extraction arm set comprises a Y-axis bearing track and a Y-axis movement mechanism, and the Y-axis bearing track comprises a bearing track main shaft and a bearing track sub-shaft;
the bearing track main shaft and the bearing track sub-shaft are disposed in parallel and symmetrically above two sides of one of the storage modules, the Y-axis movement mechanism is disposed on the bearing track main shaft and the bearing track sub-shaft and is capable of sliding along the bearing track main shaft and the bearing track sub-shaft, and the Y-axis movement mechanism is connected to the X-axis extraction arm set and is capable of driving the X-axis extraction arm set to move along the Y axis direction; and
the Y axis is perpendicular to a direction along which the storage tanks are set side by side.

18. The intelligent storage system for a sample repository according to claim 7, wherein the Y-axis extraction arm set comprises a Y-axis bearing track and a Y-axis movement mechanism, and the Y-axis bearing track comprises a bearing track main shaft and a bearing track sub-shaft;
the bearing track main shaft and the bearing track sub-shaft are disposed in parallel and symmetrically above two sides of one of the storage modules, the Y-axis movement mechanism is disposed on the bearing track main shaft and the bearing track sub-shaft and is capable of sliding along the bearing track main shaft and the bearing track sub-shaft, and the Y-axis movement mechanism is connected to the X-axis extraction arm set and is capable of driving the X-axis extraction arm set to move along the Y axis direction; and
the Y axis is perpendicular to a direction along which the storage tanks are set side by side.

19. The intelligent storage system for a sample repository according to claim 10, wherein the grabbing apparatus comprises a cryopreservation tube extraction mechanism, a storage rack grabbing mechanism, and a connecting mechanism;
an upper end of the connecting mechanism is connected to the X-axis extraction arm set, and a lower end of the connecting mechanism is connected to the storage rack grabbing mechanism and the cryopreservation tube extraction mechanism; and
the X-axis extraction arm set is capable of grabbing a cryopreservation tube or a storage rack in the storage tank and is capable of driving the cryopreservation tube or the storage rack to move along the X axis, and the Y-axis extraction arm set is capable of allowing for grabbing a cryopreservation tube or a storage rack in the storage tank and is capable of driving the cryopreservation tube or the storage rack to move along the Y axis.

20. The intelligent storage system for a sample repository according to claim 11, wherein the grabbing apparatus comprises a cryopreservation tube extraction mechanism, a storage rack grabbing mechanism, and a connecting mechanism;
an upper end of the connecting mechanism is connected to the X-axis extraction arm set, and a lower end of the connecting mechanism is connected to the storage rack grabbing mechanism and the cryopreservation tube extraction mechanism; and
the X-axis extraction arm set is capable of grabbing a cryopreservation tube or a storage rack in the storage tank and is capable of driving the cryopreservation tube or the storage rack to move along the X axis, and the Y-axis extraction arm set is capable of allowing for grabbing a cryopreservation tube or a storage rack in the storage tank and is capable of driving the cryopreservation tube or the storage rack to move along the Y axis.
